(19) United States
(12) Patent Application Publication
(10) Pub. No.: US 2025/0058064 A1
(43) Pub. Date: Feb. 20, 2025

(54) VAPORIZING DEVICE WITH SLIDING COVER TO ELIMINATE LOSS OF VAPORS

(71) Applicant: Vlad Gataullin, Hallandale Beach, FL (US)

(72) Inventor: Vlad Gataullin, Hallandale Beach, FL (US)

(73) Assignee: VIVID LABS, LLC, Hallandale Beach, FL (US)

(21) Appl. No.: 18/769,846

(22) Filed: Jul. 11, 2024

Related U.S. Application Data
(60) Provisional application No. 63/533,428, filed on Aug. 18, 2023.

(51) Int. Cl. A61M 15/06; A24F 5/10; A24F 40/10; A24F 40/46; A24F 40/60; A61M 11/04; A61M 21/00; A61M 21/02; H05B 3/28

(52) U.S. Cl. CPC A61M 15/06; A24F 5/10; A24F 40/10; A24F 40/46; A24F 40/60; A61M 11/042; A61M 21/02; H05B 3/283; A61M 2021/0016; A61M 2205/582; A61M 2205/583; A61M 2205/8206

(57) ABSTRACT

A system for burning and vaporizing consumable materials while confining vapors prior to dispensing is provided. The system comprises a portable electronic vaporizing device. The device comprises a metallic shell with two parallel rails extending from a first end of the shell. The shell contains a bowl containing combustible material, a sliding cover attached to the rails, the cover when in open position situated at a far end of the rails away from the shell and away from the bowl, and a battery-powered ceramic heater within a ceramic insulation that receives a power activation signal. The system also applies heat to the bowl when the sliding cover is in open position and transforms the combustible material into vapor based on the applied heat, the vapor trapped inside the device by the cover receiving movement into closed position over the bowl.

### CROSS REFERENCE TO RELATED APPLICATION

The present non-provisional patent application is related to U.S. Provisional Patent Application No. 63/533,428 filed Aug. 18, 2023, the contents of which are incorporated herein in their entirety.

### FIELD OF THE DISCLOSURE

The present disclosure is in the field of smoking devices. More particularly, the present disclosure provides a pipe-like device with a cover on rails that slides over a bowl containing a combustible substance when the substance begins to vaporize, the action of the cover sealing vapors inside the device and concentrating the vapors for eventual expulsion through mouthpiece for the user.

### BACKGROUND

Essential oils are plant extracts made by steaming or pressing parts of a plant, for example flowers, bark, leaves or fruit, to capture compounds that produce fragrance. Several pounds of plant product may be needed to produce a single bottle of essential oil.

Aromatherapy is a practice of using essential oils for therapeutic benefit. Aromatherapy has been used for centuries. When inhaled, the scent molecules in essential oils travel from the olfactory nerves to the brain. The scent molecules particularly impact the amygdala, the emotional center of the brain.

Essential oils may also be absorbed by the skin. A massage therapist might add wintergreen to oil to help relax tight muscles. A skincare company may add lavender to bath salts to create a soothing soak. Inhaling aromas from essential oils may stimulate areas of the limbic system, an area of the brain involved in emotions, behaviors, sense of smell, and long-term memory. The limbic system is also involved in forming memories. This may explain why familiar smells can trigger memories or emotions. The limbic system also plays a role in controlling some unconscious physiological functions, such as breathing, heart rate, and blood pressure. As such, some users claim that essential oils can exert a physical effect on the body.

### DETAILED DESCRIPTION

Systems and methods described herein provide a portable device for vaporizing combustible material, for example essential oils, for user inhalation in which the device has a sliding cover to trap and concentrate vapors once created, prevent vapor escape, and enhance user enjoyment. The device, which is handheld and rectangular in shape, has twin metal rails protruding from a first end where a ceramic bowl is located. The bowl holds combustible material for heating.

As a user is placing combustible material in the bowl for heating and vaporization, the sliding cover is positioned at a far end of the rails away from the main body of the device and in open position. The cover remains in open position at the end of the rails as the heater is activated and heats to about 250 degrees Celsius.

The generated heat is applied to the bowl containing the combustible material and the material begins to boil and vaporize. The cover is then moved back toward the main body and into closed position over the bowl. This action traps the vapors so they cannot escape.

A mouthpiece is located at the opposite end of the device from the bowl. As the vapor is being produced and the cover is closed thus containing the vapors, the user may begin drawing the vapor from the device by inhaling at the mouthpiece.

The cover remains permanently on the rails and is not detached or separated from the main body of the device. The rails are anchored inside the main body but extend inside the cover which slides on the rails. The rails are visible only when the cover is in open position.

The system has a ceramic heater combined with the bowl that heats the bowl, both components insulated to prevent burning of users' hands. The system has a printed circuit board connected to the heater and controllable by an external button.

The button may be pressed a certain number of times to start the device and another amount of times to instruct the heater to begin heating and thus burning the combustible material. The user is alerted by illumination of a small light and/or vibration that that the bowl is hot enough to place combustible material if the user has not already done so.

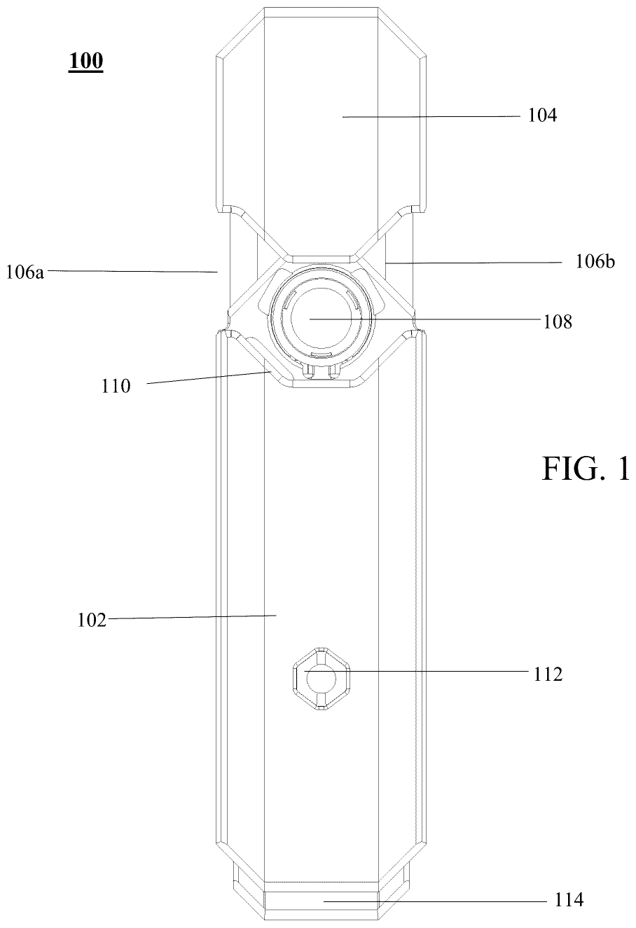

Turning to the figures, FIG. 1 is a diagram of a vaporizing device according to an embodiment of the present disclosure. FIG. 1 illustrates components and interactions of a system 100 or device 100 provided herein. System 100 comprises a case 102, a cover 104, and rails 106a-b. System 100 also comprises a bowl 108 which includes a heating element, a vapor channel 110, a button 112, and a mouthpiece 114. FIG. 1 does not depict a printed circuit board (PCB) or a battery as these components are inside the case 102.

In FIG. 1, the device 100 is in open position as the bowl 108 and the rails 106a-b are visible and the cover 104 is at the far end of the rails 106a-b. The case 102, cover 104, and rails 106a-b may be made of aluminum or other metallic material. The bowl 108 is made of ceramic as are portions of the heating element and insulation which hold heat inside the device 100 and prevents burning of users as the heating element reaches 250 degrees Celsius.

The upper surface of the case 102 has the button 112 to activate the device 100. A user may press the button 112 several times to turn the device 100 on and then presses the button 112 several times again to instruct the device 100 to start the heating element. The user may also press and hold the button 112 to begin heating the element.

When the element has reached a certain temperature, the button 112 flashes and the device 100 vibrates. These signals alert the user that the user may now slide the cover 104 along the rails 106a-b into open position such that the bowl 108 is now exposed and visible.

The user may then place the combustible substance into the bowl 108. Once the substance heats and begins to produce vapor, the cover 104 is moved into closed position over the bowl 108. Vapors are trapped inside device 100 for user inhalation via the mouthpiece 114.

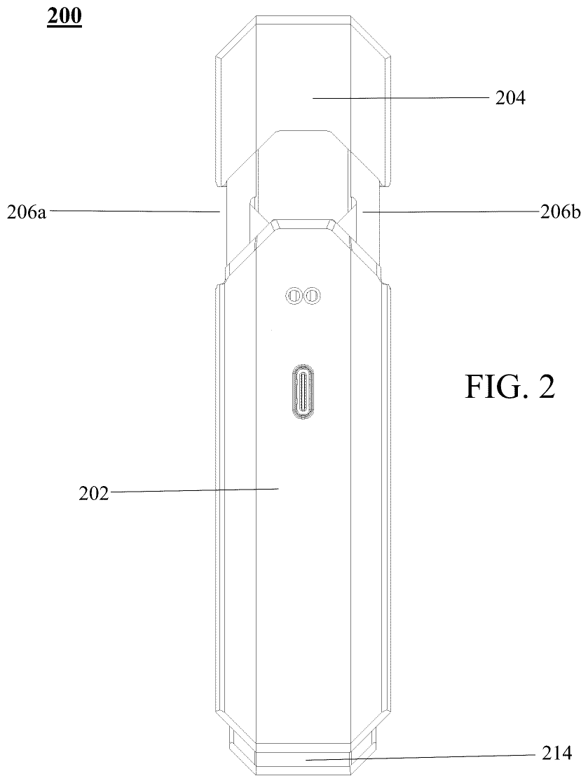

FIG. 2 is a diagram of a vaporizing device according to an embodiment of the present disclosure. Components depicted in FIG. 2 through FIG. 9 are indexed to components depicted in FIG. 1 and provided by the system 100.

FIG. 2 is a face-down or underside view of the device 200 in open position such that the rails 206a-b are visible. Also depicted in FIG. 2 are the case 202, the cover 204, and the mouthpiece 214.

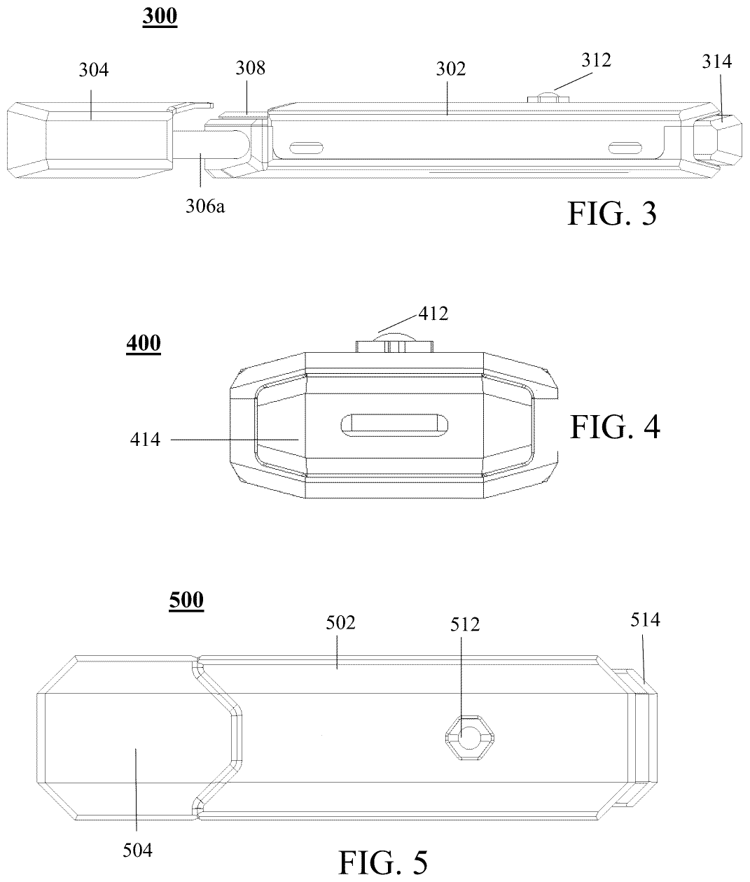

FIG. 3 is a diagram of a vaporizing device according to an embodiment of the present disclosure. FIG. 3 depicts a side view of the device 300 in open position, a view of a left side 300 of the device which includes the case 302, the cover 304, only one rail 306a, a side view of the bowl 308, the button 312, and the mouthpiece 314.

FIG. 4 is a diagram of a vaporizing device according to an embodiment of the present disclosure. FIG. 4 depicts an end view of the device 400 with only the mouthpiece 414 shown.

FIG. 5 is a diagram of a vaporizing device according to an embodiment of the present disclosure. FIG. 5 depicts a top view of the device 500 in closed state such that the bowl and rails are not visible. FIG. 5 depicts the case 502, the cover 504, the button 512, and the mouthpiece 514. As discussed above, the closed state would be in effect when the combustible material begins to vaporize, and the user intends for vapor to be trapped inside the unit. The closed state would also be in effect when the device 500 is not in use.

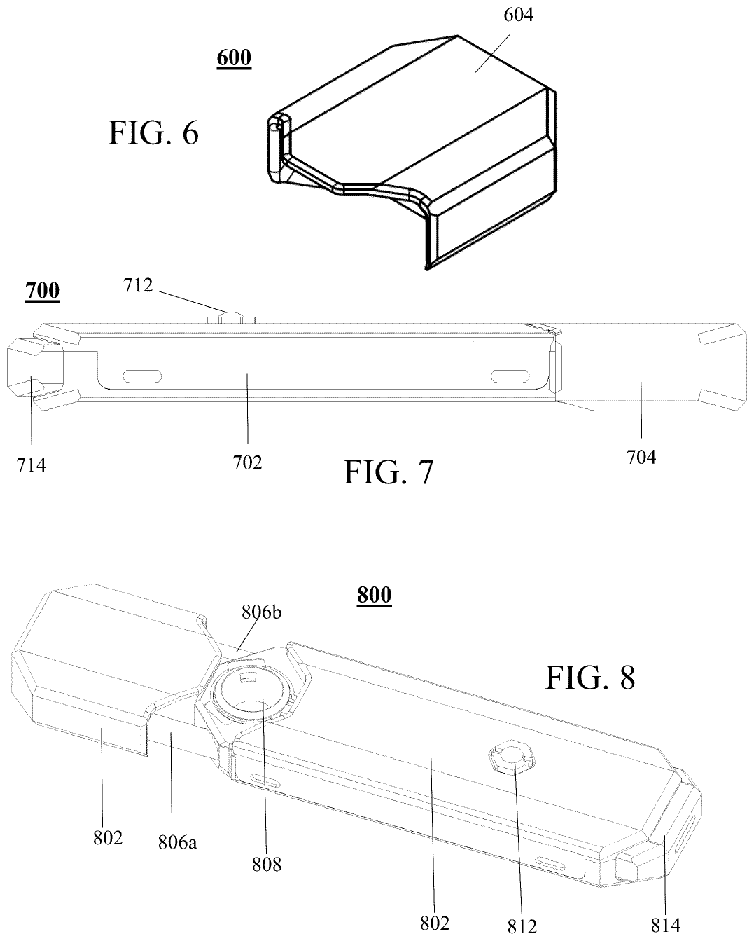

FIG. 6 is a diagram of a vaporizing device according to an embodiment of the present disclosure. FIG. 6 depicts only the cover 604. The cover 604 is shaped to slide smoothly on the rails and join tightly with the main body of the device 600 to seal over the bowl and prevent vapors from escaping and being wasted.

FIG. 7 is a diagram of a vaporizing device according to an embodiment of the present disclosure. Similar to FIG. 3, FIG. 7 depicts a side view of the device 700. But unlike FIG. 3, in FIG. 7 the device 700 is shown on its right side and is in closed position so that rails and the bowl are not visible. FIG. 7 depicts the case 702, the cover 704, the button 712, and the mouthpiece 714.

FIG. 8 is a diagram of a vaporizing device according to an embodiment of the present disclosure. FIG. 8 provides a top view of device 800 in open position depicting the case 802, the cover 804, the rails 806a-b, the bowl 808, the button 812, and the mouthpiece 814.

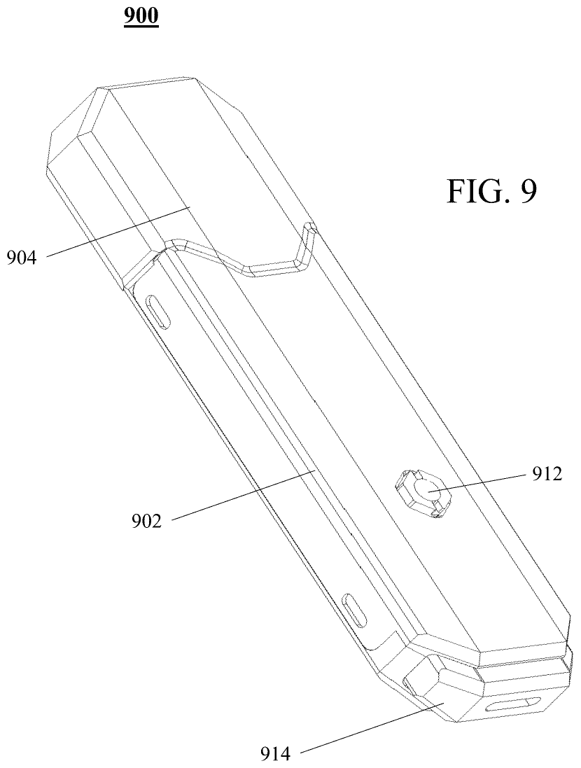

FIG. 9 is a diagram of a vaporizing device according to an embodiment of the present disclosure. FIG. 9 depicts another view of device 900 which is in closed position in this view. As with FIG. 7, which is also in closed position, FIG. 9 depicts the case 902, the cover 904, the button 912, and the mouthpiece 914.

The device 100 is directed to use with essential oil and extract for recreational aromatherapy use. The device 100 will be sold and marketed as an aromatherapy device for essential oil extract for inhalation.

In an embodiment, a system for burning and vaporizing consumable materials while confining vapors prior to dispensing is provided. The system comprises a portable electronic vaporizing device. The device comprises a metallic shell with two parallel rails extending from a first end of the shell. The shell contains a bowl containing combustible material, a sliding cover attached to the rails, the cover when in open position situated at a far end of the rails away from the shell and away from the bowl, and a battery-powered ceramic heater within a ceramic insulation that receives a power activation signal. The system also applies heat to the bowl when the sliding cover is in open position and transforms the combustible material into vapor based on the applied heat, the vapor trapped inside the device by the cover receiving movement into closed position over the bowl.

The cover receives the movement from open position to closed position when the heater commences transforming the combustible material to vapor. Closure comprises the cover receiving a sliding motion from open position in a direction toward the shell and over the bowl.

The device expels the vapors through a mouthpiece at a second end of the shell. The expulsion is further facilitated via user inhalation at the second end.

The ceramic heater heats to about 250 degrees Celsius. The device further comprises a printed circuit board and externally accessible switch for activating the ceramic heater.

In another embodiment, a system for vaporizing and dispensing material for inhalation is provided. The system comprises an electronic handheld device comprising a ceramic heater within a handheld casing. The device comprises a ceramic bowl connected to the heater and heated by the heater, the bowl located at a first end of the casing and containing combustible material. The device further comprises two parallel rails protruding from the first end below the bowl. The device also comprises a sliding cover attached to the rails that prior to the combustible material commencing conversion into vapor based on the applied heat, remains situated at a far end of the rails and away from the bowl, the situation comprising an open position. Upon the combustible material commencing vaporization, the cover receives sliding movement into a closed position over the bowl. The device also prevents, while remaining in the closed position, vapors from escaping.

The vapors, after the cover is in closed position, are drawn out of the device through a mouthpiece located at a second end of the casing opposite the first end. The ceramic heater is set within insulation.

The device contains printed circuit board (PCB) and switch for controlling the heater. The system alerts a user via visual signal and vibration when the heater has reached temperature to commence burning the combustible material. The ceramic heater heats to about 250 degrees Celsius.

In yet another embodiment, a system is provided for heating, vaporizing, and dispensing combustible substances. The system comprises a handheld device comprising a rectangular metal case, the case containing a printed circuit board (PCB), a battery, a heating element with a bowl for containing combustible substances, the bowl at a first end of the case, a mouthpiece at a second end of the case, twin rails protruding from a lower area of the first end, and a cover positioned atop the rails and slidable on the rails over the bowl and alternatively slidable away from the bowl. The cover receives movement away from the bowl and into an open position, the movement exposing the bowl to user access. Upon the bowl receiving placement of a combustible substance and upon the substance receiving heat from the element and producing vapor, the cover receives movement toward and over the element and traps vapor inside the case.

A channel inside the case promotes movement of vapor from the bowl to a mouthpiece at a second end of the case. The printed circuit board and the battery are situated inside the case and provide power to heat the element.

The heating element and bowl are ceramic. After activation the device vibrates and emits a flashing signal to advise that the heating element is heated to a level such that the combustible substance should be placed in the bowl. The element heats to about 250 degrees Celsius. The combustible substance is essential oils.

1. A system for burning and vaporizing consumable materials while confining vapors prior to dispensing, comprising:
a portable electronic vaporizing device, comprising:
a metallic shell with two parallel rails extending from a first end of the shell, the shell further containing:
a bowl containing combustible material;
a sliding cover attached to the rails, the cover when in open position situated at a far end of the rails away from the shell and away from the bowl; and
a battery-powered ceramic heater within a ceramic insulation that:
receives a power activation signal,
applies heat to the bowl when the sliding cover is in open position, and
transforms the combustible material into vapor based on the applied heat, the vapor trapped inside the device by the cover receiving movement into closed position over the bowl.

2. The system of claim 1, wherein the cover receives the movement from open position to closed position when the heater commences transforming the combustible material to vapor.

3. The system of claim 1, wherein closure comprises the cover receiving a sliding motion from open position in a direction toward the shell and over the bowl.

4. The system of claim 1, wherein the device expels the vapors through a mouthpiece at a second end of the shell.

5. The system of claim 4, wherein the expulsion is further facilitated via user inhalation at the second end.

6. The system of claim 1, wherein the ceramic heater heats to about 250 degrees Celsius.

7. The system of claim 1, wherein the device further comprises a printed circuit board and externally accessible switch for activating the ceramic heater.

8. A system for vaporizing and dispensing material for inhalation, comprising:
an electronic handheld device comprising:
a ceramic heater within a handheld casing;
a ceramic bowl connected to the heater and heated by the heater, the bowl located at a first end of the casing and containing combustible material;
two parallel rails protruding from the first end below the bowl;
a sliding cover attached to the rails that:
prior to the combustible material commencing conversion into vapor based on the applied heat, remains situated at a far end of the rails and away from the bowl, the situation comprising an open position,
upon the combustible material commencing vaporization, receives sliding movement into a closed position over the bowl, and
prevents, while remaining in the closed position, vapors from escaping.

9. The system of claim 8, wherein the vapors, after the cover is in closed position, are drawn out of the device through a mouthpiece located at a second end of the casing opposite the first end.

10. The system of claim 8, wherein the ceramic heater is set within insulation.

11. The system of claim 8, wherein the device contains printed circuit board (PCB) and switch for controlling the heater.

12. The system of claim 8, wherein the system alerts a user via visual signal and vibration when the heater has reached temperature to commence burning the combustible material.

13. The system of claim 8, wherein the ceramic heater heats to about 250 degrees Celsius.

14. A system for heating, vaporizing, and dispensing combustible substances, comprising:
a handheld device comprising:
a rectangular metal case, the case containing:
a printed circuit board (PCB),
a battery,
a heating element with a bowl for containing combustible substances, the bowl at a first end of the case,
a mouthpiece at a second end of the case,
twin rails protruding from a lower area of the first end; and
a cover positioned atop the rails and slidable on the rails over the bowl and alternatively slidable away from the bowl that:
receives movement away from the bowl and into an open position, the movement exposing the bowl to user access,
upon the bowl receiving placement of a combustible substance and upon the substance receiving heat from the element and producing vapor, receives movement toward and over the element, and
traps vapor inside the case.

15. The system of claim 14, wherein a channel inside the case promotes movement of vapor from the bowl to a mouthpiece at a second end of the case.

16. The system of claim 14, wherein the printed circuit board and the battery are situated inside the case and provide power to heat the element.

17. The system of claim 14, wherein the heating element and bowl are ceramic.

18. The system of claim 14, wherein after activation the device vibrates and emits a flashing signal to advise that the heating element is heated to a level such that the combustible substance should be placed in the bowl.

19. The system of claim 14, wherein the element heats to about 250 degrees Celsius.

20. The system of claim 14, wherein the combustible substance is essential oils.
